(19) United States
(12) Patent Application Publication
(10) Pub. No.: US 2012/0100589 A1
(43) Pub. Date: Apr. 26, 2012

(54) METHOD FOR METHANE GENERATION

(75) Inventors: Danielle Rhine, Roanoke, VA (US); Anders Peter Jensen, Arhus C. (DK); Stefan Borre-Gude, Skanderborg (DK)

(73) Assignees: XERGI A/S, Stoevring (DK); NOVOZYMES A/S, Bagsvaerd (DK)

(21) Appl. No.: 13/255,338

(22) PCT Filed: Mar. 8, 2010
(86) PCT No.: PCT/US10/26540

§ 371 Date: Jan. 5, 2012

Related U.S. Application Data
(60) Provisional application No. 61/158,720, filed on Mar. 9, 2009.

(51) Int. Cl. C12P 5/02; C12P 1/02; C12P 1/00; C12P 1/04; C12N 1/20; C12N 1/14

(52) U.S. Cl. 435/167; 435/252.4; 435/252.1; 435/256.1; 435/252.5; 435/253.3; 435/41; 435/170; 435/171

(57) ABSTRACT

A method for treatment of a material comprising lignocellulosic fibres is disclosed. More particularly, the treatment increases the accessibility of the lignocellulosic fibres for following microbial or biological processes.

### REFERENCE TO A DEPOSIT OF BIOLOGICAL MATERIAL

This application contains a reference to a deposit of biological material, which deposit is incorporated herein by reference. For complete information see last page of the description.

### FIELD OF THE INVENTION

The present invention relates to a process for treatment of a material comprising lignocellulosic fibres which treatment increases the degradability of the lignocellulosic fibres. In particular the invention relates to methane production from manure, preferably manure derived from cattle, where the treatment of the invention is used to increase the methane production in comparison with untreated manure.

### BACKGROUND OF THE INVENTION

Most natural plant based material comprises a significant amount of lignocellulosic fibres that are undigestible or only slowly digestible in many biological systems. This has the consequence that for many biological processes converting plant based material a significant fraction of the treated material will not be digested or only digested in a low degree during the treatment.

For example in a usual biogas production plant manure is fermented under anaerobic conditions forming biogas and a waste material consisting to a large extent of lignocellulosic fibres that is hardly digested at all under to conditions of an anaerobic biogas process.

In areas with concentrated animal production, manure can often cause environmental problems. These include odor formation, pollution of waterways and the creation of infertile land. As worldwide animal production continues to increase so does the environmental impact. At the same time, manure is largely an unexploited renewable energy source, in particular the production of biogas such as methane.

The generation of biogas from manure is an old technology and today production facilities range from simple covered lagoons to sophisticated industrial plants with controlled process parameters. The industrial manure based plants of today have a low return on investment (ROI) due to the low energy intensity of raw manure (a combination of urine and feces) combined with the relatively large capital expenditure needed to erect a biogas plant. The use of this technology is typically limited unless the biogas or electricity production is subsidized (e. g. in Germany). Due to low conversion of the lignocellulose present in the manure (currently achieving up to approximately 50% of theoretical methane production potential for dairy cow manure), high energy materials are commonly added to obtain additional biogas. Such materials include high energy crops or food processing waste. However, it is estimated that the limited availability and expense of high energy waste can limit the application of biogas extraction to only 5% of the available manure.

It would be beneficial to provide a method for enhancing methane production from materials comprising lignocellulosic fibres, such as manure from dairy cattle or other livestock. This technology can facilitate a global change in manure management practices and turn an environmental problem into a profitable and environmentally beneficial solution.

### SUMMARY OF THE INVENTION

In the first aspect the invention relates to a method for treatment of a material comprising lignocellulosic fibres comprising the steps of:

a. providing a material comprising lignocellulosic fibres;

b. inoculating the material from step a with one or more microorganisms; and

c. incubating the material under aerobic conditions.

The method according to this aspect increases the degradability of the lignocellulosic fibres making them more accessible for a following microbial or biological process such as for example a biogas production process leading to a higher yield than would have been possible without the treatment of the invention.

In a preferred embodiment, the invention relates to a method for generating methane from a material comprising lignocellulosic fibres, preferably manure, further comprising:
a. providing a material comprising lignocellulosic fibres;
   1. performing a first anaerobic fermentation of the material from step a for generation of a first amount of methane;
   2. following step 1, optionally separating a fraction comprising fibres;
b. inoculating the material of step 1 or 2 with one or more microorganisms;
c. incubating the inoculated material from step b under aerobic conditions; and
d. performing a second anaerobic fermentation of the material obtained in step c, for generation of a second amount of methane.

The method according to this embodiment provides for a higher yield of methane compared to traditional biogas methods typically comprising only a first anaerobic fermentation step. Thus, according to the invention, a considerably higher amount of biogas can be produced based on the same amount of starting material.

In a second aspect the invention relates to a method for selecting a microorganism or a mixture of two or more microorganisms capable of digesting the lignocellulosic fibre fraction comprising the steps of:
i) providing a material comprising lignocellulosic fibres;
ii) incubating the material comprising lignocellulosic fibres provided in step i. with a candidate microorganism or a mixture of two or more microorganisms under aerobic conditions;
iii) analysing the fibre fraction obtained in step ii to determine whether a part of the lignocellulosic fibres have been made accessible by the treatment.

The material comprising lignocellulosic fibres is, in this aspect, preferably derived from manure that has been subjected to an anaerobic fermentation for biogas production by a fractionation process providing a fraction comprising lignocellulosic fibres.

In a third aspect the invention provides a convenient method for selecting microorganisms suitable for the method of the invention.

In a fourth aspect the invention provides a microorganism or a mixture of microorganisms that are particular suited for the method according to the invention.

In a fifth aspect the invention relates to the use of a microorganism or a mixture of two or more microorganisms according to the fourth aspect in a method according to the invention.

### SHORT DESCRIPTION OF THE DRAWINGS

FIG. 1. A methane calibration curve with concentrations ranging from 1×10⁻⁷ to 3.8×10⁻⁶ mol CH₄, which includes standards bracketing the methane concentrations obtained in the samples.

FIG. 2. Methane production curves for the fibres treated for 3 (FIG. 2a), 7 (FIG. 2b) and 14 days (FIG. 2c) (Example 1). Methane levels from fibres treated with water alone is included as a reference.

FIG. 3. Methane production curves for the fibres treated for 9 (FIG. 3a), and 15 days (FIG. 3b) (Example 2). Methane levels from fibres treated with water alone for 15 days is included as a reference.

FIG. 4. Methane production curves showing the impact on methane production of the dose of microbial product added during the aerobic treatment of the fibre for 28 days (Example 3). Methane levels from fibres treated with water alone is included as a reference.

### DETAILED DESCRIPTION OF THE INVENTION

### Definitions:

The term “biogas” is according to the invention intended to mean the gas obtained in a conventional anaerobic fermentor using manure. The main component of biogas is methane and the terms “biogas” and “methane” are in this application and claims used interchangeably.

The term “primary digester” is in this application and claims intended to mean the container wherein the first anaerobic fermentation takes place.

The term “secondary digester” is in this application and claims intended to mean the container wherein the second anaerobic fermentation takes place. Depending on the particular configuration of the biogas facility the primary digester may also serve as the secondary digester.

In the method for treatment according to the first aspect of the invention the material is usually provided in a container, even thought the treatment of the invention may be conduction without a container such as e.g. in a pile.

The material comprising lignocellulosic fibres may be any treated or untreated plant material as well as any composition comprising such plant material.

The plant material may be treated or untreated. Treated plant material is in this application and claims intended to mean any suitable treatment of the plant material e.g. the plant material may be comminuted using suitable techniques such as mincing, chopping and cutting; or heated using e.g. steam treatment or boiling, etc.

Material comprising lignocellulosic fibres may according to the invention be any material comprising lignocellulosic fibres. Examples of such materials include, but are not limited to, wood, straw, hay, grass, silage, such as cereal silage, corn silage, grass silage; bagasse and manure such as manure from livestock e.g. cattle, cows, poultry, pigs, sheep and horses. A preferred material comprising lignocellulosic fibres is manure, preferably manure from cattle, most preferably from dairy cows.

Hay and straw contain nearly 90% w/w lignocellulose. In cattle manure, the lignocellulose fibres (crude fibre) make up 40-50% of the total solids. The lignocellulose fibres are made up of a core of carbohydrates, in particular cellulose and hemicellulose, which makes up 63-78% of the fibre structure. The cellulose and hemicellulose are packed and supported by lignin which makes up 15-38% of the lignocellulose structure. A study to compare the composition of a variety of dairy cattle and pig manure has shown that on average, cattle manure contains: VFA (Volatile Fatty Acids) (36 g/kg VS), protein (150 g/kg VS), lipids (69 g/kg VS), degradable carbohydrates (434 g/kg VS), non-degradable carbohydrates (191 g/kg VS), lignin (121 g/kg VS), and crude fiber (270 g/kg VS). Pig manure contains on average: VFA (30 g/kg VS), protein (202 g/kg VS), lipids (163 g/kg VS), degradable carbohydrates (390 g/kg VS), non-degradable carbohydrates (148 g/kg VS), lignin (68 g/kg VS), and crude fiber (171 g/kg VS).

The term lignocellulosic fibres is in this application and claims intended to mean any plant material comprising lignocellulose, lignin and/or cellulose in any form, amounts and ratios. The lignocellulosic fibres may further comprise other plant derived components such as starch, glucans, arabans, galactans, pectins, mannans, galactomannans and hemicelluloses such as xylans.

The microorganisms according to the invention may be selected among bacteria, yeasts or fungi, or mixtures thereof. The microorganisms or mixtures of two or more microorganisms according to the invention has the benefit that they provide for a high amount of methane production in the second anaerobic fermentation step of the method according to the first aspect of the invention. Thus, the use of the microorganisms according to the invention in a method according to the invention provides for a surprisingly high production of methane. Preferred examples of microorganisms according to the invention includes strains of the genus: Bacillus, Pseudomonas, Enterobacter, Rhodococcus, Acinetobacter, and Aspergillus such as Bacillus licheniformis, Pseudomonas putida, Enterobacter dissolvens, Pseudomonas fluorescens, Rhodococcus pyridinivorans, Acinetobacter baumanii, Bacillus amyloliquefaciens, Bacillus pumilus, Pseudomonas plecoglossicida, Pseudomonas pseudoacaligenes, Pseudomonas antarctica, Pseudomonas monteilii, Pseudomonas mendocina, Bacillus subtilis, Aspergillus niger and Aspergillus oryzae and any combinations or two or more thereof.

Particular preferred strains include: Bacillus subtilis (NRRL B-50136), Pseudomonas monteilii (NRRL B-50256), Enterobacter dissolvens (NRRL B-50257), Pseudomonas monteilii (NRRL B-50258), Pseudomonas plecoglossicida (ATCC 31483), Pseudomonas putida (NRRL B-50247), Pseudomonas plecoglossicida (NRRL B-50248), Rhodococcus pyridinivorans (NRRL 50249), Pseudomonas putida (ATCC 49451), Pseudomonas mendocina (ATCC 53757), Acinetobacter baumanii (NRRL B-50254), Bacillus pumilus (NRRL B-50255), Bacillus licheniformis (NRRL B-50141), Bacillus amyloliquefaciens (NRRL B-50151), Bacillus amyloliquefaciens (NRRL B-50019), Pseudomonas mendocina (ATCC 53757), Pseudomonas monteilii (NRRL B-50250), Pseudomonas monteilii (NRRL B-50251), Pseudomonas monteilii (NRRL B-50252), Pseudomonas monteilii (NRRL B-50253), Pseudomonas antarctica (NRRL B-50259), Bacillus amyloliquefaciens (ATCC 55405), Aspergillus niger (NRRL 50245), and Aspergillus oryzae (NRRL 50246).

The skilled person will appreciate how to determine suitable amounts of these preferred strains in uses according to the invention, using well known techniques. In preferred embodiments the strains are added in amounts in the range of 1.0×10⁶ to 5.0×10⁹ CFU/g.

As examples of particular preferred microorganisms or mixtures of two or more microorganisms can be mentioned:
A mixture containing: Bacillus subtilis (NRRL B-50136; 1.1×10⁹ CFU/g), Pseudomonas monteilii (NRRL B-50256; 0.6×10⁹ CFU/g), Enterobacter dissolvens (NRRL B-50257; 0.6×10⁹ CFU/g), Pseudomonas monteilii (NRRL B-50258; 0.8×10⁹ CFU/g), Pseudomonas fluorescens (ATCC 31483; 0.8×10⁹ CFU/g), Pseudomonas putida (NRRL B-50247; 0.4×10⁹ CFU/g), Pseudomonas plecoglossicida (NRRL B-50248; 0.4×10⁹ CFU/g), Rhodococcus pyridinivorans (NRRL 50249; 0.8×10⁹ CFU/g), Pseudomonas putida (ATCC 49451, 0.4×10⁹ CFU/g), Pseudomonas mendocina (ATCC 53757; 0.8×10⁹ CFU/g), and Acinetobacter baumanii (NRRL B-50254; 0.2×10⁹ CFU/g;
A mixture containing: Bacillus subtilis (NRRL B-50136; 1.6×10⁹ CFU/g), Bacillus pumilus (NRRL B-50255; 0.2×10⁹ CFU/g), Bacillus amyloliquefaciens (NRRL B-50141; 0.2×10⁹ CFU/g), Bacillus amyloliquefaciens (NRRL B-50151; 0.2×10⁹ CFU/g), Bacillus amyloliquefaciens (NRRL B-50019; 0.2×10⁹ CFU/g), Pseudomonas monteilii (NRRL B-50256; 0.2×10⁹ CFU/g), Enterobacter dissolvens (NRRL B-50257; 0.3×10⁹ CFU/g), Pseudomonas monteilii (NRRL B-50258; 0.8×10⁹ CFU/g), Pseudomonas plecoglossicida (ATCC 31483; 0.7×10⁹ CFU/g), Pseudomonas putida (NRRL B-50247; 0.2×10⁹ CFU/g), Pseudomonas plecoglossicida (NRRL B-50248; 0.2×10⁹ CFU/g), Rhodococcus pyridinivorans (NRRL 50249; 0.3×10⁹ CFU/g), Pseudomonas putida (ATCC 49451; 0.2×10⁹), Pseudomonas mendocina (ATCC 53757; 0.3×10⁹ CFU/g), Pseudomonas monteilii (NRRL B-50250; 0.1×10⁹ CFU/g), Pseudomonas monteilii (NRRL B-50251; 0.1×10⁹ CFU/g), Pseudomonas monteilii (NRRL B-50252; 0.1×10⁹ CFU/g), Pseudomonas monteilii (NRRL B-50253; 0.1×10⁹ CFU/g), and Pseudomonas antarctica (NRRL B-50259; 0.2×10⁹CFU/g); and
A mixture containing: Bacillus subtilis (NRRL B-50136; 3.5×10⁹ CFU/g), Bacillus amyloliquefaciens (ATCC 55405; 1.0×10⁹ CFU/g), Pseudomonas antarctica (NRRL B-50259; 0.2×10⁹ CFU/g), Aspergillus niger (NRRL 50245; 0.8×10⁹ CFU/g), and Aspergillus oryzae (NRRL 50246; 0.8×10⁹ CFU/g).

Further, the microorganism or mixture of two or more microorganisms commercially available from Novozymes Biological Inc. under the trade names: BI-CHEM ABR-Hydrocarbon, BI-CHEM DC 1008 CB and Manure Degrader are also suitable according to the third aspect of the invention.

The incubation under aerobic conditions may be performed as batch process, fed batch process or continuous process. In a batch process the container is filled, a suitable inoculum of the microorganisms is added and the process proceeding for a desired time. In a fed batch process a initial volume of material comprising lignocellulosic fibres is added into the container, typically 25-75% of the total operational volume of the container, a suitable inoculum of the microorganism is added and the process is proceeding until a certain conversion/cell density is reached where additional feed in form of material comprising lignocellulosic fibres is added at a suitable rate and the process is continued until the container is full and optionally for an additional time without additional feed. In a continuous process the process is started by adding material comprising lignocellulosic fibres into the container and a suitable inoculum of the microorganism is added, when a desired cell density is reached a stream of the composition in the container is removed and simultaneously a stream of material comprising lignocellulosic fibres is added to the container so that the volume remains essentially constant and the process is continued in principle as long as desired. It may even be possible to use a combination of these techniques. These techniques are known within the art and the skilled person will appreciate how to find suitable parameters for a particular process depending on the particular dimensions and properties of the container.

Means for aeration are well known in the art and it is within the capabilities of the skilled person to select suitable means for aeration for the present invention. Usually aeration is performed by blowing atmospheric air through the composition typically via one or more tube(s) or pipe(s) located in the lower part of the container said one or more tube(s) or pipe(s) is/are provided with holes at regular intervals to provide for an even distribution of the air in the composition. Other means for aerating may also be used according to the invention.

The rate of aeration during the aerobic fermentation step is selected to provide for a convenient growth rate of the microorganisms. Rate of aeration may be measured in volume air per volume ferment per minute (v/v/m) and usually aeration in the range of 0.01 v/v/m to 10 v/v/m is suitable, preferably 0.05 v/v/m to 5 v/v/m, more preferred 0.1 v/v/m to 2 v/v/m, more preferred 0.15 v/v/m to 1.5 v/v/m and most preferred 0.2 v/v/m to 1 v/v/m.

The duration of this step will be decided taking into account that on one side the incubation under aerobic conditions should be continued for a sufficient long time to make a satisfactory part of the lignocellulosic soluble and available for the following microbial or biological process, on the other side the aerobic step should not be extended so long that a too large fraction of the fibre fraction is combusted. Usually the aerobic fermentation is continued for 5 to 30 days, preferably from 7 to 25 days, more preferred from 10 to 20 days and most preferred around 15 days. It has been found that using such an incubation period a suitable high fraction of the lignocellulosic fibres is converted into a form that can be converted in a following microbial or biological process.

The temperature in this step should be selected taking into account the particular requirements of the microorganism or mixture of two or more microorganisms used according to the invention. Usually the temperature is selected in the range of 10° C. to 60° C., preferably in the range of 15° C. to 50° C., more preferred in the range of 20° C. to 45° C., even more preferred in the range of 25° C. to 40° C. and most preferred about 35° C.

The method according to the invention increases the degradability of the lignocellulosic fibres making them more accessible for a following microbial or biological process such as for example a biogas production process leading to a higher yield than would have been possible without the method of the invention.

The incubation under aerobic conditions is continued until the degradability of the lignocellulosic fibres has been increased in a satisfactory extent so that a considerable high fraction of lignocellulosic fibres has been made accessible for a following microbial or biological process.

When lignocellulosic fibres have been made accessible according to the present invention the accessible fibres or part thereof will be available for the following microbial or biological process, meaning that the accessible fibres or part thereof can be converted in the following microbial or biological process. Thus, it can be determined if the accessability of lignocellulosic fibres have been increased by the method of the invention by performing a following microbial or biological process on the material treated according to the invention and comparing the yield of said following microbial or biological process with a corresponding following microbial or biological process using same material comprising lignocellulosic fibres but without the method of the invention.

Using biogas formation as an example of a following microbial or biological process it can be determined if the method for treatment according to the invention increases the accessibility of a material comprising lignocellulosic fibres, a material comprising lignocellulosic fibres can be treated using a method of the invention, followed by a usual anaerobic biogas forming process and the yield of the biogas using the material comprising lignocellulosic fibres treated according to the invention can be determined and compared with the same biogas forming process but without the method of the invention. If the yield of biogas is higher using the method of the invention, accourding to the invention, the accessibility of the lignocellulosic fibres has increased.

Another method for determining if the accessibility of the lignocellulosic fibres has been increased is determination of the amount of soluble carbohydrates after the method of the invention has been performed, for example, as shown in Experimental methods sections 1-3. The skilled person will appreciate that the increased accessibility according to the invention can be determined in other ways using different following microbial or biological methods.

The method according to the invention may be used in connection with any following microbial or biological process where it is desired to achieve an increased utilisation of the material comprising lignocellulosic fibres. The invention may be used but not limited to the following microbial or biological processes: biogas formation and feed for live stocks.

In one embodiment a method of the invention relates to the production of methane. In this embodiment the production of methane may be conducted as a two step process comprising a method according to the invention followed by a process for biogas production, which in principle may be any process for biogas formation as known within the area.

In another embodiment, the production of methane may be conducted as a process comprising a first process for biogas formation, followed by a method according to the invention, again followed by a second process for biogas formation.

In another embodiment, the material comprising lignocellulosic fibres is preferably manure such as manure which may be manure produced in any livestock. Typically the manure is derived from cattle, pigs, horses, sheep, chicken or goats, where manure derived from cattle is preferred, in particular manure from dairy cattle.

This embodiment is further explained with reference to manure, however, it should be appreciated that it is not limited to manure.

The first anaerobic fermentation in the method corresponds to the traditional fermentation of manure for the production of biogas. Thus this step may in principle be performed using techniques known in the art for fermenting manure for the generation of biogas. The first anaerobic fermentation step takes place in a suitable container as it is known within the art.

In this step the fermentation is conducted using known technology until the gas production ceases to an unacceptable low rate. The skilled person will be able to select a suitable end point for this fermentation.

Separation of a fraction comprising fibres may be also performed using techniques known in the art for separation of compositions having rheological properties similar to fermented manure leaving a biogas fermentation process. These techniques for separation are known in the art and it is within the skills of the ordinary practitioner to select suitable parameters for the separation process.

After the aerobic fermentation the fermented manure is fermented anaerobically using a process that in principle is identical to the first anaerobic fermentation, and in this step an additional amount of biogas is produced.

This second anaerobic fermentation may in principle take place in the same container wherein the aerobic fermentation has taken place by simply stopping the aeration and adding a suitable amount of inoculums.

Alternatively, in particular for continuous processes, the manure leaving the aerobic fermentation is transferred to another container wherein the second anaerobic fermentation takes place. This container may be the same container wherein the first anaerobic fermentation takes place or it may be a separate container.

The amount of methane obtained by the process depends on the composition of the manure, which again depends on the animals from which the manure is derived, the feed they are given etc.; but typically an amount of methane of approximately 225 ml CH₄/g VS is achieved in the first anaerobic fermentation. The second anaerobic fermentor typically provides at least 10%, preferably at least 25%, more preferred at least 30%, more preferred at least 35%, more preferred at least 40%, more preferred at least 45%, even more preferred at least 50%, most preferred at least 55% and in a particular preferred embodiment at least 60% of the amount of biogas obtained in the first anaerobic fermentation.

In the second aspect of the invention a method for selecting a microorganism or a mixture of two or more microorganisms suitable for enhancing the methane potential of the fibre fraction from the first anaerobic fermentation is provided.

The fibre fraction for use in this aspect of the invention may in principle be prepared similar to the fibre fraction provided in the method according to the first aspect of the invention. However, the first aspect of the invention relates to a method performed on large scale (production scale), typically several m³, whereas the second aspect of the invention typically will be performed in much smaller scale such as the scale that typically is used in a laboratory such as in the order of a few g or kg.

When the fibre fraction has been provided a candidate microorganism or mixture of two or more microorganisms are incubated with an aliquot of the fibre fraction, typically a few grams even though the actual amount used in this step is not essential for the invention. In addition nutrients, salts, vitamins and other growth factors necessary to support the growth of the candidate microorganism or mixture of two or more microorganism may be added.

The incubation of candidate microorganism or mixture of two or more microorganism with the fibre faction is typically for a period of for 5 to 30 days, preferably from 7 to 25 days, more preferred from 10 to 20 days and most preferred around 15 days.

### EXAMPLES

Experimental Methods

1. Aerobic Treatment of Fibre from Primary Digester

In 400-ml beakers, 50 g of fibre were combined with 70 ml of deionized water and 5 ml of the microbial consortium to a final concentration of ˜5×10⁵ cfu/ml. The water and microbial consortium were thoroughly mixed within the fibre. Each beaker was covered with steam paper to allow gas exchange, and they were weighed at the start of each experiment. The covered beakers were incubated at 30° C. for up to 2 weeks. Periodically, the beakers were re-weighed and water lost through evaporation was added back.

2. Anaerobic Methane Production Potential

Following the aerobic treatment, 4 g of treated fibre from each condition were transferred to separate 500 ml amber bottles. To each amber bottle, 200 ml of anaerobic digestate inoculum was added, and the headspace was flushed with N₂ gas. Prior to use, fresh anaerobic digestate inoculum was incubated at 52° C. for at least 2 weeks to remove residual methane potential and to lower background methane production. The bottles were stoppered and sealed to prevent gas loss and to retain anaerobiosis. Duplicate bottles of each treatment and of relevant controls were incubated at 52° C. for 28 days. Samples of the headspace were periodically taken using a locking 1-ml gas tight syringe, transferred to gas tight GC vials, and analyzed for methane content using a gas chromatograph.

3. Gas Chromatography (GC) Method

Methane was measured using a Shimadzu GC-2010 gas chromatograph with a flame ionization detector (GC-FID) and a SupeIQPLOT 30 m×0.53 m column (Supelco). Using a He carrier gas flow rate of 30 ml/min, and injection port, oven and detector temperatures of 100° C., 35° C. and 235° C., respectively, the methane peak had a retention time of 2.7±0.2 minutes. Calibration curves were generated by injecting different concentrations of methane from a 49% methane standard.

4. Description of Microbial Consortia

Consortium 1 contains: Bacillus subtilis (NRRL B-50136; 1.1×10⁹ CFU/g), Pseudomonas monteilii (NRRL B-50256; 0.6×10⁹ CFU/g), Enterobacter dissolvens (NRRL B-50257; 0.6×10⁹ CFU/g), Pseudomonas monteilii (NRRL B-50258; 0.8×10⁹ CFU/g), Pseudomonas plecoglossicicida (ATCC 31483; 0.8×10⁹ CFU/g), Pseudomonas putida (NRRL B-50247; 0.4×10⁹ CFU/g), Pseudomonas plecoglossicicida (NRRL B-50248; 0.4×10⁹ CFU/g), Rhodococcus pyridinivorans (NRRL 50249; 0.8×10⁹ CFU/g), Pseudomonas putida (ATCC 49451, 0.4×10⁹ CFU/g), Pseudomonas mendocina (ATCC 53757; 0.8×10⁹ CFU/g), and Acintobacter baumanii (NRRL B-50254; 0.2×10⁹CFU/g).

Consortium 2 contains: Bacillus subtilis (NRRL B-50136; 1.6×10⁹ CFU/g), Bacillus pumilus (NRRL B-50255; 0.2×10⁹ CFU/g), Bacillus licheniformis (NRRL B-50141; 0.2×10⁹ CFU/g), Bacillus amyloliquefaciens (NRRL B-50151; 0.2×10⁹ CFU/g), Bacillus amyloliquefaciens (NRRL B-50019; 0.2×10⁹ CFU/g), Pseudomonas monteilii (NRRL B-50256; 0.2×10⁹ CFU/g), Enterobacter dissolvens (NRRL B-50257; 0.3×10⁹ CFU/g), Pseudomonas monteilii (NRRL B-50258; 0.8×10⁹ CFU/g), Pseudomonas plecoglossicicida (ATCC 31483; 0.7×10⁹ CFU/g), Pseudomonas putida (NRRL B-50247; 0.2×10⁹ CFU/g), Pseudomonas plecoglossicicida (NRRL B-50248; 0.2×10⁹ CFU/g), Rhodococcus pyridinivorans (NRRL 50249; 0.3×10⁹ CFU/g), Pseudomonas putida (ATCC 49451; 0.2×10⁹), Pseudomonas mendocina (ATCC 53757; 0.3×10⁹ CFU/g), Pseudomonas monteilii (NRRL B-50250; 0.1×10⁹ CFU/g), Pseudomonas monteilii (NRRL B-50251; 0.1×10⁹ CFU/g), Pseudomonas monteilii (NRRL B-50252; 0.1×10⁹ CFU/g), Pseudomonas monteilii (NRRL B-50253; 0.1×10⁹ CFU/g), and Pseudomonas antarctica (NRRL B-50259; 0.2×10⁹ CFU/g).

Consortium 3 contains: Bacillus subtilis (NRRL B-50136; 3.5×10⁹ CFU/g), Bacillus amyloliquefaciens (ATCC 55405; 1.0×10⁹ CFU/g), Pseudomonas antarctica (NRRL B-50259; 0.2×10⁹ CFU/g), Aspergillus niger (NRRL 50245; 0.8×10⁹ CFU/g), and Aspergillus oryzae (NRRL 50246; 0.8×10⁹CFU/g).

### Example 1

Successful microbial conversion of the residual fibre from the original anaerobic treatment was assessed by measuring the amount of additional methane obtained in a batch type anaerobic digester following the aerobic microbial treatment. This aerobic treatment of the fibre with selected microbial strains is referred to as “aerobic treatment” or “treatment”.

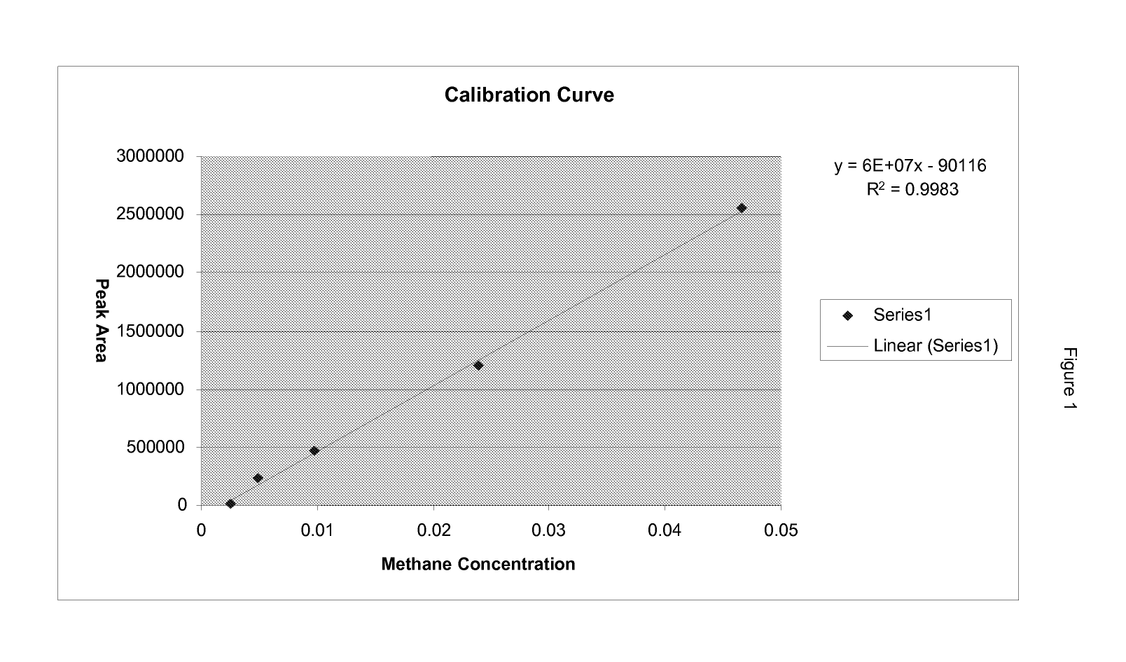

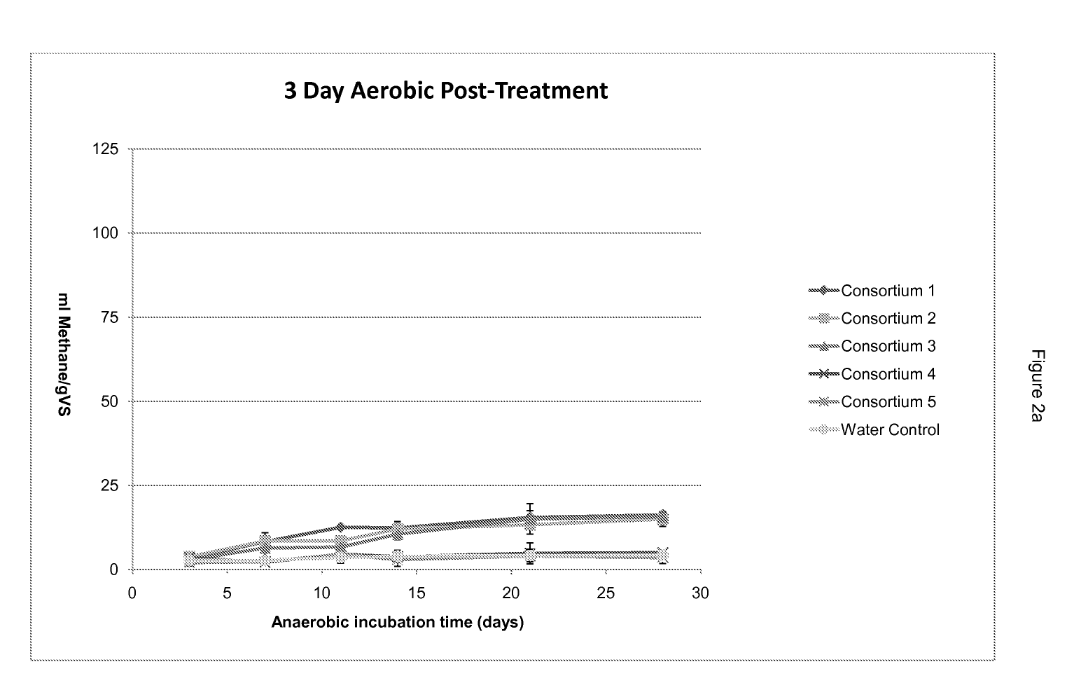

First, a standard calibration curve was derived which utilized methane concentrations from 1×10⁻⁷ to 3.8×10⁻⁶ mol CH₄, and which bracket the concentrations of methane obtained in typical samples (FIG. 1). The linear curve obtained has an R² value of 0.9983.

In this first example, residual fibre from the anaerobic digestion process was treated with one of the described microbial consortia (1, 2, or 3) at 30° C. for 3, 7 or 14 days. Fibre from all treated samples were added to anaerobic digestion bottles as described. At indicated times methane generation was quantified from both microbially treated samples and controls, as described. This is compared with methane generated from fiber treated with water only. The addition of water did not significantly enhance methane production.

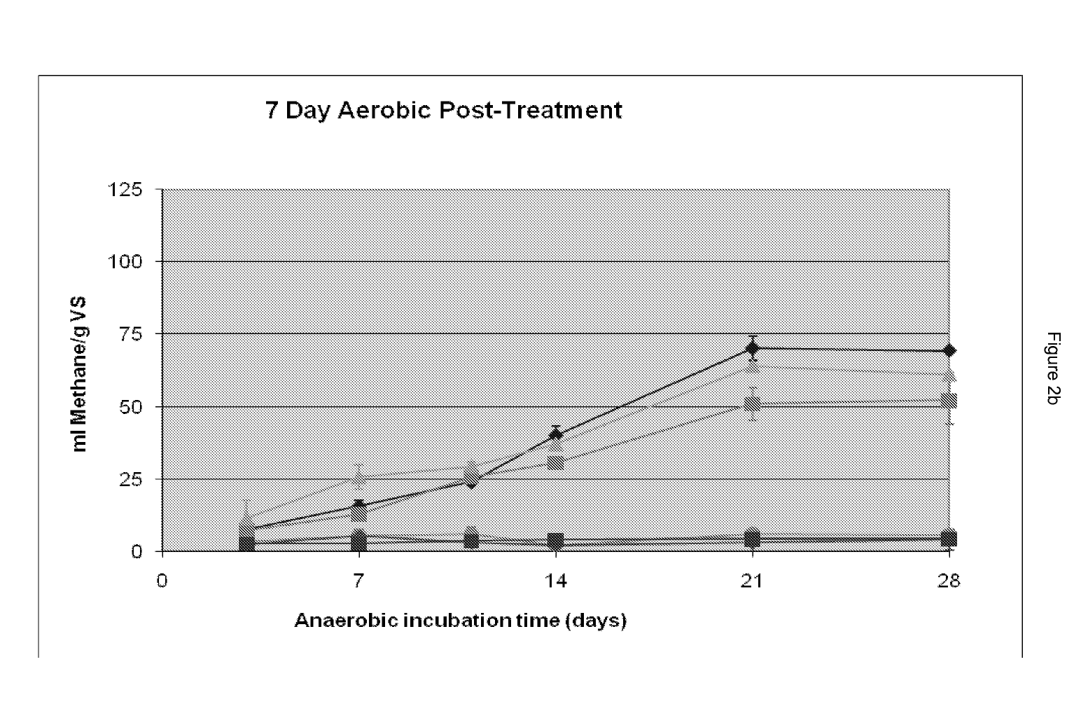

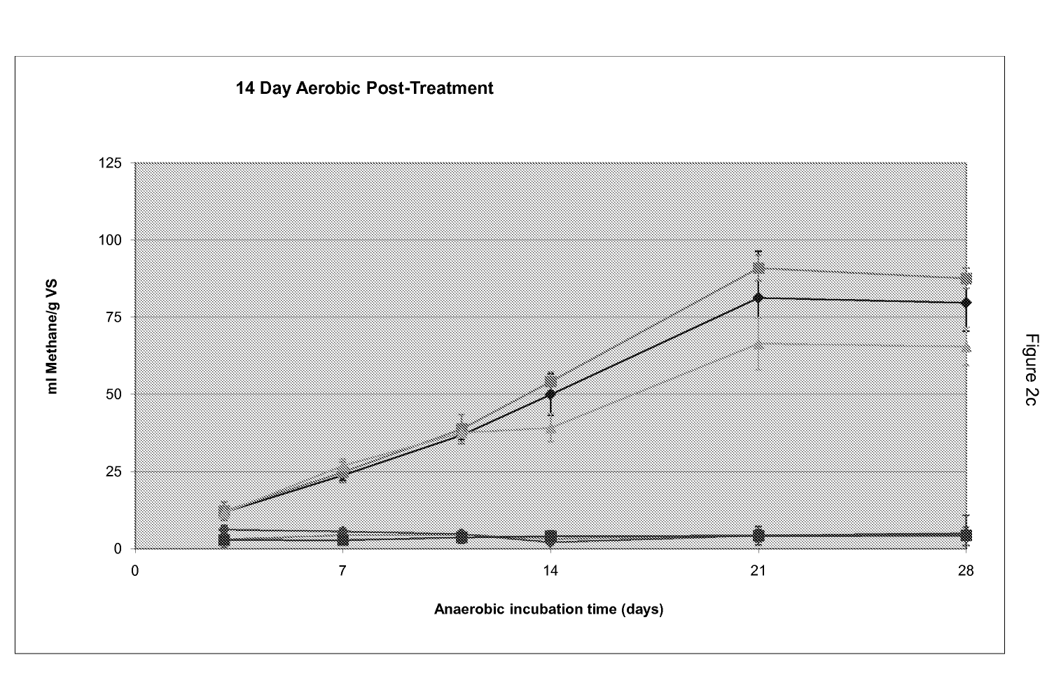

Fibre treated with three different microbial consortiums (1, 2, and 3) resulted in an increase of methane produced during the 28 day anaerobic incubation period (FIG. 2). When treated with the microbial products for 3 days, methane production of 15-16.2 ml CH₄/g VS was observed. Three separate aerobic bottles for each fibre treatment were assessed to provide the standard deviations given. Continued enhancement in methane production occurred with increased incubation time and the maximum methane production obtained was observed for fiber post-treated for 2 weeks.

Overall methane production in fiber treated with the three microbial consortia (1, 2, and 3) were 80 to 100 ml additional CH₄/g VS over that derived from the water treated fibre controls after 14 days of aerobic post-treatment.

### Example 2

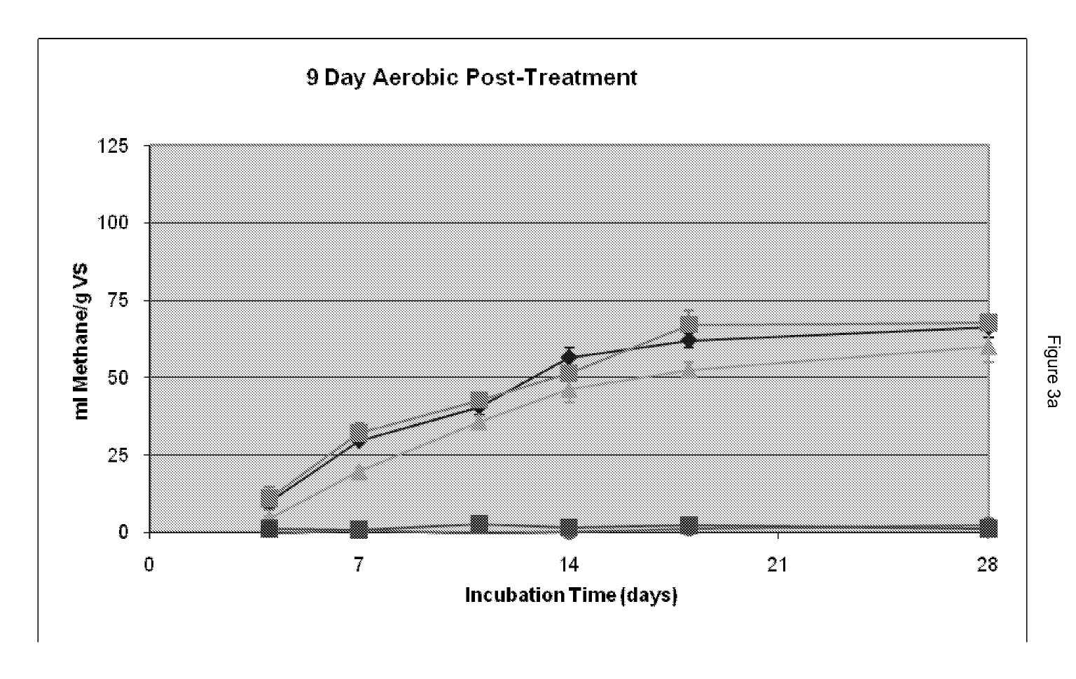

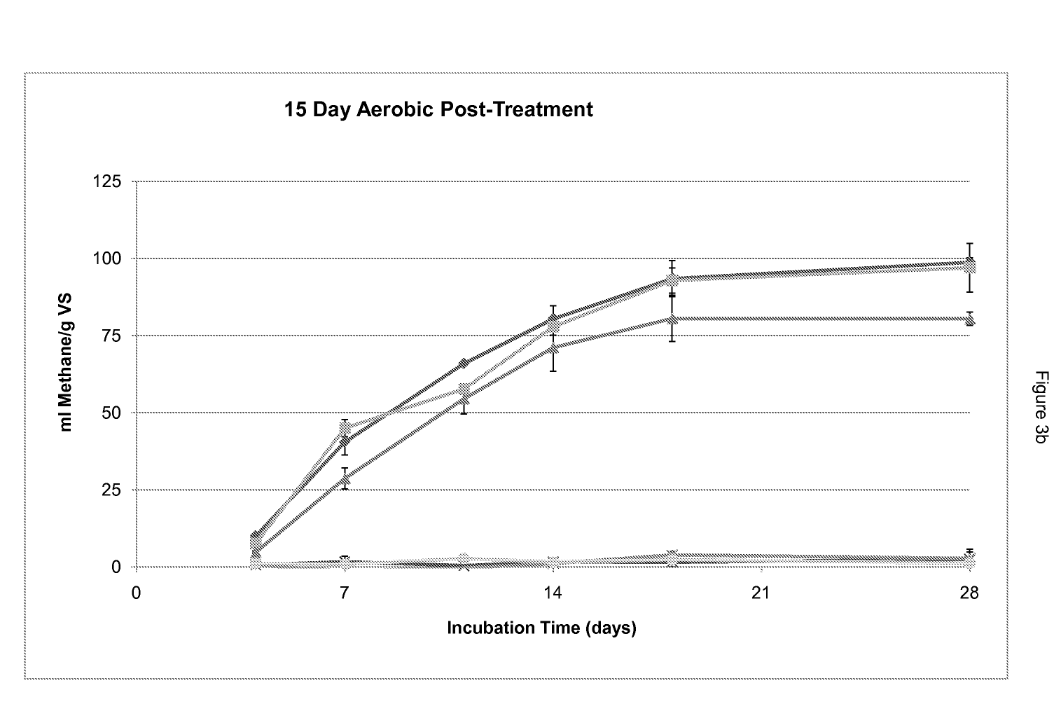

A second study was completed to confirm the activity of the microbial Consortia, now evaluating the effects of 9-day and 15-day aerobic treatement. The results (FIG. 3) confirm the ability of Consortia 1, 2, and 3 to enhance methane recovery from treated fiber. All methods used are as described in Example 1. The greatest methane generation in Example 2 is achieved after 15 days of aerobic composting.

### Example 3

In this experiment the impact on methane production of the dose of microbial product added during the aerobic treatment of the fibre was studied. The aerobic treatment of the fibre was conducted for two weeks at 30° C. as described above, but microbial products of Consortium 1, Consortium 2 and Consortium 3 as described above, were added in doses of 5*10⁴, 5*10⁵ and 5 *10⁶ cfu/ml. After aerobic treatment 4 g of the microbially treated fibre was analyzed for anaerobic methane production potential. Methane production was measured after 1, 2, 3 and 4 weeks of incubation at 52° C. In the negative control, the experiment without addition of microorganisms the methane production was less than 5 ml/g VS.

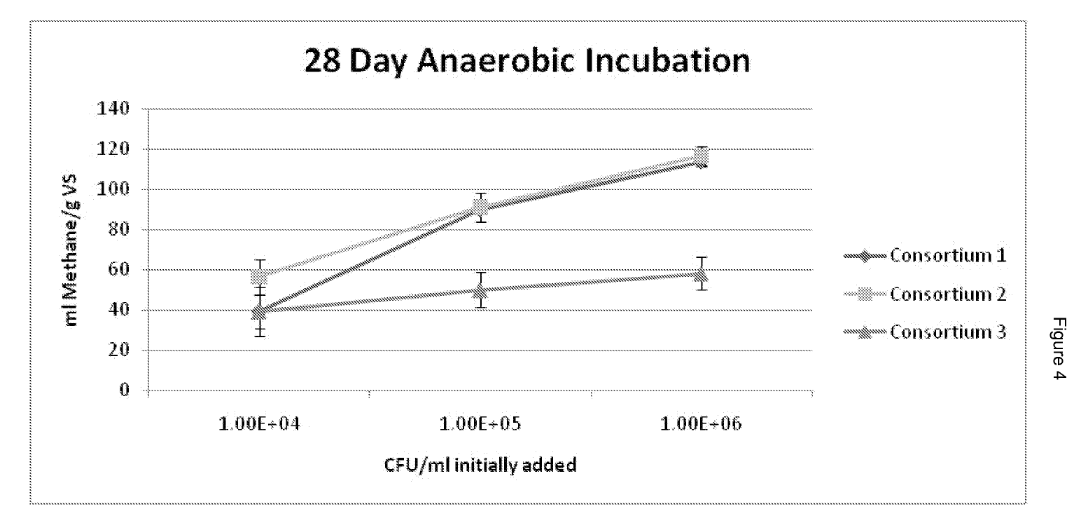

Results of this experiment are shown in FIG. 4. The curves show the highest methane production obtained during the four weeks. The results show that the methane production potential is increased with increasing doses of micro-organisms in the range from 10⁴ to 10⁶ CFU/ml.

### Example 4

In this experiment the microbial treatment was performed at two independent laboratories. The treated fibre was exchanged between the laboratories. The aerobic treatment of fibre was conducted for two weeks at 30° C. with the addition of 5*10⁵ cfu/ml of three different microbial product variants of Consortium 1 as described above. In both laboratories the same microbial products were tested and the tested fibre was the same.

After aerobic treatment 4 g of fibre was analyzed for anaerobic methane production potential. Methane production was measured after 1, 2, 3 and 4 weeks of incubation at 52° C. The two independent laboratories used anaerobic inoculum that were pre-incubated for at 2-3 weeks at 52° C. prior to the start of the determination of anaerobic methane production potential. The anaerobic inoculum was from two different and completely independent biogas plants. The inoculum used in laboratory 1 was from a plant situated in the USA, the inoculum in laboratory 2 was from a Danish biogas plant. Negative controls to measure the inherent background methane potential from each anaerobic digestate source were also included.

Table 1 shows the results from this experiment. These results show significant methane enhancement with all of the microbial aerobic treatments conducted at both laboratories. However, significant CH₄ enhancement above the negative control was only observed with the anaerobic methane potential studies conducted in laboratory 1. The higher amount of methane produced in all samples, including the negative controls, tested in the anaerobic digestate source used in lab 2 suggests that the inherent background methane potential from the digestate was significantly higher than that associated with the anaerobic digestate used in lab 1. These results suggest that the aerobic microbial treatment may have limitations depending on the efficiency of the anaerobic system, the substrate source, handling and other additional processing of the substrate prior to addition to the anaerobic digester.

Table 1. Methane production per gram Volatile Solids (VS) in experiments conducted by two independent laboratories. For results in Lab1/Lab1 column both the aerobic treatment and the anaerobic methane production potential were conducted in laboratory 1. For results in Lab2/Lab1 aerobic treatment was performed in Lab 2 and anaerobic methane production potential was evaluated in Lab 1. For Lab2/Lab2 the whole experiment was conducted in laboratory 2. For Lab1/Lab2 aerobic treatment was conducted in Lab 1 and anaerobic methane production potential was determined in Lab 2.

| | ml Methane/g VS | | | |
|---|---|---|---|---|
| | Lab1/Lab1 | Lab2/Lab1 | Lab2/Lab2 | Lab1/Lab2 |
| Consortium 1-A | 177 | 175 | 156 | 162 |
| Consortium 1-B | 170 | 182 | 146 | 178 |
| Consortium 1-C | 255 | 210 | 155 | 192 |
| Negative Control | 71 | 94 | 170 | 202 |

### Example 5

In this experiment the microbial treatment was evaluated on anaerobic digestate obtained from different sources. Two samples were from anaerobic digesters run under mesophilic conditions (37° C.), the other two were from thermophilic (52° C.) anaerobic digesters, but all anaerobic systems were used for digesting dairy cattle manure in the US. Following a 2 week aerobic microbial treatment at 30° C. with two consortia (5*10⁵ cfu/ml initial rate), 4 g of the treated fibre was analyzed for methane production potential in each of the four different anaerobic digestate samples. The samples obtained from mesophilic conditions were incubated for 6 weeks at 37° C., and the samples from the thermophilic anaerobic digesters were incubated for 4 weeks at 52° C.

The data presented in Table 2 shows similar methane enhancement with both microbial consortia compared to the negative control in the thermophilic anaerobic digestate samples (Thermophilic 1 and 2). A greater methane enhancement compared to the negative control was also observed in one of the mesophilic samples (Mesophilic 1), but no significant enhancement was observed in the other mesophilic sample (Mesophilic 2). This study shows that this aerobic microbial treatment can work in both types of anaerobic digestion systems (mesophilic and thermophilic). Based on the lack of enhancement observed with one of the mesophilic digestate samples, there could still be differences in the substrates that may limit the microbial treatment even if all the substrates were from dairy cattle manure.

Table 2. Methane production per gram Volatile Solids (VS) in experiments conducted with anaerobic digestate from two thermophilic and two mesophilic systems digesting dairy cattle manure. The results are presented as total ml CH₄ per g VS produced from microbially treated fiber incubated anaerobically. The thermophilic samples were incubated at 52° C. for 4 weeks and the mesophilic samples were incubated for 6 weeks at 37° C.

| | ml Methane/g VS | | | |
|---|---|---|---|---|
| | Mesophilic 1 | Thermophilic 1 | Mesophilic 2 | Thermophilic 2 |
| Consor- | 263 | 94 | 281 | 109 |
| tium | | | | |
| 1-A | | | | |
| Consor- | 225 | 105 | 260 | 114 |
| tium | | | | |
| 1-B | | | | |
| Negative | 187 | 67 | 272 | 55 |
| Control | | | | |

### Deposit of Biological Material

The following biological material has been deposited under the terms of the Budapest Treaty with the Agricultural Research Service Patent Culture Collection (NRRL), Northern Regional Research Center, 1815 University Street, Peoria, Ill., 61604, USA, and given the following accession number:

| Deposit | Accession Number | Date of Deposit |
|---|---|---|
| Aspergillus niger | NRRL 50245 | Feb. 18, 2009 |
| Aspergillus oryzae | NRRL 50246 | Feb. 18, 2009 |
| Pseudomonas putida | NRRL B-50247 | Feb. 18, 2009 |
| Pseudomonas plecoglossicida | NRRL B-50248 | Feb. 18, 2009 |
| Rhodococcus sp. | NRRL 50249 | Feb. 18, 2009 |
| Pseudomonas monteilii | NRRL B-50250 | Feb. 18, 2009 |
| Pseudomonas monteilii | NRRL B-50251 | Feb. 18, 2009 |
| Pseudomonas monteilii | NRRL B-50252 | Feb. 18, 2009 |
| Pseudomonas monteilii | NRRL B-50253 | Feb. 18, 2009 |
| Acinetobacter baumanii | NRRL B-50254 | Feb. 18, 2009 |
| Bacillus pumilus | NRRL B-50255 | Feb. 18, 2009 |
| Pseudomonas monteilii | NRRL B-50256 | Feb. 18, 2009 |
| Enterobacter dissolvens | NRRL B-50257 | Feb. 18, 2009 |
| Pseudomonas monteilii | NRRL B-50258 | Feb. 18, 2009 |
| Pseudomonas Antarctica | NRRL B-50259 | Feb. 18, 2009 |
| Bacillus subtilis | NRRL B-50136 | May 30, 2008 |
| Pseudomonas plecoglossicida | ATCC 31483 | Commercial |
| Pseudomonas putida | ATCC 49451 | Commercial |
| Pseudomonas mendocina | ATCC 53757 | Commercial |
| Bacillus licheniformis | NRRL B-50141 | Jun. 3, 2008 |
| Bacillus amyloliquefaciens | NRRL B-50151 | Jul. 11, 2008 |
| Bacillus amyloliquefaciens | NRRL B-50019 | Mar. 14, 2007 |
| Bacillus pumilus | NRRL B-50016 | Mar. 14, 2007 |

The strains have been deposited under conditions that assure that access to the culture will be available during the pendency of this patent application to one determined by foreign patent laws to be entitled thereto. The deposits represent a substantially pure culture of the deposited strain. The deposits are available as required by foreign patent laws in countries wherein counterparts of the subject application or its progeny are filed. However, it should be understood that the availability of a deposit does not constitute a license to practice the subject invention in derogation of patent rights granted by governmental action.

1-14. (canceled)

15. A method for treatment of a material comprising lignocellulosic fibers comprising the steps of:
a. providing a material comprising lignocellulosic fibers;
b. inoculating the material from step a with one or more microorganisms; and
c. incubating the material under aerobic conditions.

16. The method of claim 15, wherein the material comprising lignocellulosic fibers is selected from the group consisting of corn silage, grass silage, bagasse, and manure.

17. The method of claim 16, wherein the manure is selected from cows, poultry, pigs or other livestock.

18. The method of claim 15, wherein the one or more microorganisms are selected from the group consisting of Acinetobacter, Aspergillus, Bacillus, Enterobacter, Pseudomonas, and Rhodococcus, or any combination of two or more thereof.

19. The method of claim 18, wherein the one or more microorganisms are selected from the group consisting of Acinetobacter baumanii, Aspergillus niger, Aspergillus oryzae, Bacillus amyloliquefaciens, Bacillus licheniformis, Bacillus pumilus, Bacillus subtilis, Enterobacter dissolvens, Pseudomonas antarctica, Pseudomonas fluorescens, Pseudomonas mendocina, Pseudomonas monteilii, Pseudomonas plecoglossicida, Pseudomonas pseudoalcaligenes, Pseudomonas putida, and Rhodococcus pyridinivorans, or any combination of two or more thereof.

20. The method of claim 18, wherein each strain is added in an amount in the range of 1.0×10⁶ to 5.0×10⁹ CFU/g.

21. The method of claim 15 further comprising an anaerobic fermentation for biogas production taking place after step c.

22. The method of claim 21, further comprising an anaerobic fermentation for biogas production taking place before step b.

23. The method of claim 15, wherein the incubation under aerobic conditions in step c is for a predetermined amount of time or until a desired degree of degradation of the material comprising lignocellulosic fibers has been achieved.

24. A method for generating methane from a material comprising lignocellulosic fibers, comprising:
(a) providing a material comprising lignocellulosic fibers;
(1) performing a first anaerobic fermentation of the material from step a for generation of a first amount of methane;
(2) following step 1, optionally separating a fraction comprising fibers;
(b) inoculating the material of step 1 or 2 with one or more microorganisms;
(c) incubating the inoculated material from step b under aerobic conditions; and
(d) performing a second anaerobic fermentation of the material obtained in step c, for generation of a second amount of methane.

25. The method of claim 24, wherein the material comprising lignocellulosic fibers is derived from manure that has been subjected to a biogas production process including a fractionation process providing a fraction comprising lignocellulosic fibers.

26. A microorganism or a mixture of two or more microorganisms capable of enhancing the methane potential of a lignocellulosic fiber fraction selected from the group consisting of Acinetobacter baumanii (NRRL B-50254), Aspergillus niger (NRRL 50245), Aspergillus oryzae (NRRL 50246), Bacillus amyloliquefaciens (ATCC 55405), Bacillus amyloliquefaciens (NRRL B-50019), Bacillus amyloliquefaciens (NRRL B-50151), Bacillus licheniformis (NRRL B-50141), Bacillus pumilus (NRRL B-50255), Bacillus subtilis (NRRL B-50136), Enterobacter dissolvens (NRRL B-50257), Pseudomonas antarctica (NRRL B-50259), Pseudomonas mendocina (ATCC 53757), Pseudomonas monteilii (NRRL B-50250), Pseudomonas monteilii (NRRL B-50251), Pseudomonas monteilii (NRRL B-50252), Pseudomonas monteilii (NRRL B-50253), Pseudomonas monteilii (NRRL B-50256), Pseudomonas monteilii (NRRL B-50258), Pseudomonas plecoglossicida (ATCC 31483), Pseudomonas plecoglossicida (NRRL B-50248), Pseudomonas putida (ATCC 49451), Pseudomonas putida (NRRL B-50247), and Rhodococcus pyridinivorans (NRRL 50249), or any combination thereof.

27. The microorganism or mixture of microorganisms of claim 26, wherein each microorganism is present in an amount in the range of 1.0×10⁶ to 5.0×10⁹ CFU/g.

28. A mixture of microorganisms as claimed in claim 27, wherein the mixture is:
(a) a mixture of microorganisms comprising Acinetobacter baumanii (NRRL B-50254; 0.2×10⁹ CFU/g), Bacillus subtilis (NRRL B-50136; 1.1×10⁹ CFU/g), Enterobacter dissolvens (NRRL B-50257; 0.6×10⁹ CFU/g), Pseudomonas fluorescens (ATCC 31483; 0.8×10⁹ CFU/g), Pseudomonas monteilii (NRRL B-50256; 0.6×10⁹ CFU/g), Pseudomonas monteilii (NRRL B-50258; 0.8×10⁹ CFU/g), Pseudomonas plecoglossicida (NRRL B-50248; 0.4×10⁹ CFU/g), Pseudomonas putida (ATCC 49451, 0.4×10⁹ CFU/g), Pseudomonas putida (NRRL B-50247; 0.4×10⁹ CFU/g), Pseudomonas mendocina (ATCC 53757; 0.8×10⁹ CFU/g), and Rhodococcus pyridinivorans (NRRL 50249; 0.8×10⁹CFU/g);
(b) a mixture of microorganisms comprising Bacillus amyloliquefaciens (NRRL B-50019; 0.2×10⁹CFU/g), Bacillus amyloliquefaciens (NRRL B-50151; 0.2×10⁹CFU/g), Bacillus licheniformis (NRRL B-50017; 0.2×10⁹ CFU/g), Bacillus pumilus (NRRL B-50255; 0.2×10⁹ CFU/g), Bacillus subtilis (NRRL B-50136; 1.6×10⁹ CFU/g), Enterobacter dissolvens (NRRL B-50257; 0.3×10⁹ CFU/g), Pseudomonas antarctica (NRRL B-50259; 0.2×10⁹ CFU/g), Pseudomonas mendocina (ATCC 53757; 0.3×10⁹ CFU/g), Pseudomonas monteilii (NRRL B-50250; 0.1×10⁹ CFU/g), Pseudomonas monteilii (NRRL B-50251; 0.1×10⁹ CFU/g), Pseudomonas monteilii (NRRL B-50252; 0.1×10⁹ CFU/g), Pseudomonas monteilii (NRRL B-50253; 0.1×10⁹ CFU/g), Pseudomonas monteilii (NRRL B-50256; 0.2×10⁹ CFU/g), Pseudomonas monteilii (NRRL B-50258; 0.8×10⁹ CFU/g), Pseudomonas plecoglossicida (ATCC 31483; 0.7×10⁹ CFU/g), Pseudomonas plecoglossicida (NRRL B-50248; 0.2×10⁹ CFU/g), Pseudomonas putida (ATCC 49451; 0.2×10⁹), Pseudomonas putida (NRRL B-50247; 0.2×10⁹ CFU/g), and Rhodococcus pyridinivorans (NRRL 50249; 0.3×10⁹CFU/g),;
(c) a mixture of microorganisms comprising Aspergillus niger (NRRL 50245; 0.8×10⁹ CFU/g), Aspergillus oryzae (NRRL 50246; 0.8×10⁹ CFU/g), Bacillus amyloliquefaciens (ATCC 55405; 1.0×10⁹ CFU/g), Bacillus subtilis (NRRL B-50136; 3.5×10⁹ CFU/g), and Pseudomonas antarctica (NRRL B-50259; 0.2×10⁹CFU/g); or
(d) the mixture of microorganisms in the commercially available products marketed under the trade names: BI-CHEM ABR-Hydrocarbon, BI-CHEM DC 1008 CB and Manure Degrader.
